(19) United States
(12) Patent Application Publication
(10) Pub. No.: US 2010/0095959 A1
(43) Pub. Date: Apr. 22, 2010

(54) DEVICE FOR RAMPED CONTROL OF CPAP TREATMENT

(76) Inventor: Michael FARRELL, Bella Vista, NSW (AU)

Correspondence Address: GOTTLIEB RACKMAN & REISMAN PC, 270 MADISON AVENUE, 8TH FLOOR, NEW YORK, NY, 10016-0601, US

(21) Appl. No.: 12/445,543

(22) PCT Filed: Oct. 16, 2007
(86) PCT No.: PCT/US07/81502

§ 371 Date: Apr. 23, 2009

Related U.S. Application Data
(60) Provisional application No. 60/829,699, filed on Oct. 17, 2006.

(51) Int. Cl. A61M 16/06

(52) U.S. Cl. 128/202.13; 600/300

(57) ABSTRACT

A CPAP apparatus intended to operate between a predetermined minimum pressure and maximum therapeutic pressure including: a variable pressured air source and means to vary the air pressure delivered therefrom, an apparatus for sealed air communication with a patient's respiratory system, an air communication line from the air source to the apparatus; a transducer adapted to detect the presence of a triggering event by the patient's respiratory system; such as snoring sounds, rate of breathing, inhaled air flow volume, and inhaled air flow rate and a feedback system controlling the output pressure of the air source in response to an output from the transducer so as to increase the output air pressure from said air source, in response to a triggering event, in accordance with a predefined procedure.

### FIELD OF THE INVENTION

The present invention relates to the treatment of partial or complete upper airway occlusion, a condition where the upper airway collapses, particularly under the reduced pressure generated by inhalation. This is most likely to happen during unconsciousness, sleep or anesthesia.

### BACKGROUND OF THE INVENTION

A particular application of the present invention is to the treatment of snoring and sleep apnea. Sleep apnea is characterized by complete occlusion of the upper airway passage during sleep while snoring is characterized by partial occlusion. Obstructive sleep apnea sufferers repeatedly choke on their tongue and soft palate throughout an entire sleep period resulting in lowered arterial blood oxygen levels and poor quality of sleep. It should be realized that although the following specification discusses sleep apnea in detail, the present invention also applies to the treatment of other forms of upper airway disorders.

The application of continuous positive airway pressure (CPAP) has been used as a means of treating the occurrence of obstructive sleep apnea. See WO 82/03548. The patient is connected to a positive pressure air supply by means of a nose mask or nasal prongs. The air supply breathed by the patient, is at all times at slightly greater than atmospheric pressure. For example, gauge pressures will typically be within the range of 2 cm to 25 cm. The application of continuous positive airway pressure provides what can be described as a “pneumatic splint”, supporting and stabilizing the upper airway and thus eliminating the occurrence of upper airway occlusions. It is effective in eliminating both snoring and obstructive sleep apnea and in many cases, is effective in treating central and mixed apnea.

U.S. Pat. No. 6,705,315 described a CPAP device that was more acceptable to the patient before and during initial sleep by operating at an initially low pressure but automatically increasing to an appropriate therapeutic pressure before apnea occurs. That invention provided increases in treatment pressure in response to certain events, typically snoring. Its main concern, however, was keeping the pressure as low as required. Thus it provided for the reduction of treatment pressure in the absence of events indicating a continuing breathing problem. It was not concerned with bringing the patient to a pressure level sufficient to treat all the occurring breathing problems and leaving the pressure at that level for the remainder of a treatment session. Furthermore where an appropriate treatment level was determined clinically, the invention of the '315 patent did not assure that the patient would be brought to that level, either in response to breathing events or on a preplanned schedule over several days that raised the achieved pressure level by steps to a predetermined therapeutic level.

### BRIEF DESCRIPTION OF THE INVENTION

The present invention operates in one of two modes. In the first, so-called CPAP/APAP hybrid mode, the CPAP pressure starts each session at a predetermined minimum pressure. During the course of the breathing session, over a period of hours, the pressure is increased by a predetermined step at each occurrence of a triggering event and remains at that pressure until the occurrence of the next triggering event, provided that the step never causes the pressure to exceed a predetermined maximum pressure that is bounded above by a predetermined therapeutic pressure. In a second mode, the so-called acclimate mode, the patient begins each days session at a predetermined minimum pressure. The pressure is then increased according to a ramp function to achieve a daily pressure that advances from day to day until it achieves a predetermined therapeutic pressure. It should be understood that the term day to day could refer some other period of time than a day such as a half day.

Preferably, a feedback control is cooperative with a variable speed air compressor of the CPAP apparatus, the processor regulating the speed of the compressor when in use by increasing speed in response to a signal equivalent to a preprogrammed signal indicative of a predetermined snoring pattern. The said signal could also be indicative of a predetermined pattern in other respirator parameters.

The CPAP pressure in the CPAP/APAP mode is thus increased in response to triggering physiological events, particularly the onset of snoring, which was detected by a sound transducer. A feedback device for a CPAP apparatus includes a variable speed air compressor, a nose piece for sealed air communication with a patient's respiratory system, an air line from the compressor to the nose piece, an enclosed microphone connected to the air line so as to be in sound communication with the patient's respiratory system, and a feedback system controlling the speed of the air compressor in response to an output from the microphone so as to increase compressor speed in response to detected sound indicative of heavy snoring in accordance with a pre-defined procedure.

In another form of the apparatus there is provided a CPAP apparatus including a variable speed air compressor, a nose piece for sealed air communication with a patient's respiratory system, an air line from the compressor to the nose piece, a pressure transducer connected to the air line so as to be in pressure communication with the patient's respiratory system, and a feedback system controlling the speed of the air compressor in response to an output or outputs from the pressure transducer so as to increase compressor speed in response to detected patterns of sound or respiratory parameters indicative of snoring or breathing disorders in accordance with a predefined procedure. That procedure is to start each session at a predetermined minimum pressure and then to increase the pressure by equal steps in response to each triggering event and to hold the pressure at that value until the occurrence of the next triggering event.

The invention also provides a variable speed air compressor and control system in the CPAP apparatus, the control system regulating the speed of the compressor when in use by increasing its speed in accordance with a predefined procedure whereby the commencement of operation of the compressor occurs at a preselected minimum speed with a gradually increasing compressor speed over a preselected period of time to a preselected maximum speed. In this “acclimate” mode the pressure starts each day from a predetermined minimum value and proceeds by a ramp to a value for that day, such that the value reached each day increases by step to a predetermined pressure that is bounded by the therapeutic pressure.

The CPAP/APAP embodiment of the invention provides an advantage in that the patient is exposed to a comfortably low pressure before falling asleep and during initial stages of sleep while the necessary increased pressure is reached by the time it is required or alternatively, in the acclimate mode, the patient is gradually brought over a period of days to that therapeutic pressure.

### BRIEF DESCRIPTION OF THE DRAWINGS

The present invention will now be described in detail by way of reference to the attached drawings in which:

FIG. 1a is a prior art snoring detection apparatus for use with the present invention;

FIG. 1b is a schematic sectional view of another prior art snoring detection apparatus in accordance for use with the present invention;

FIG. 2a depicts the CPAP/APAP mode of pressure increases of the present invention.

FIG. 2b depicts the acclimate mode of pressure increases of the present invention.

FIG. 3 is a diagram of a further embodiment of the present invention.

FIG. 4 is a circuit diagram of the device of FIG. 3.

FIG. 5 is a diagram of an embodiment of another aspect of the invention.

FIG. 6 is a circuit diagram of the device of FIG. 5.

FIG. 7 shows the different pressure waves generated by the extraneous high frequency noise associated with air flow from the blower and the pressure waves of interest indicative of snoring and breathing and the composite wave comprising the contribution of all sources of pressure wave;

FIG. 8 shows how the composite wave from the pressure sensor is filtered to generate information on the parameters of interest;

FIG. 9 shows in schematic form how any or all of these parameters can be used either alone or by reference to control data to provide a signal to a Motor Speed Controller;

FIG. 10 shows in schematic form how the computing system which analyzes the parameters of interest with reference to control patient data controls motor speed and air pressure delivered to the patient.

### PREFERRED EMBODIMENTS OF THE INVENTION

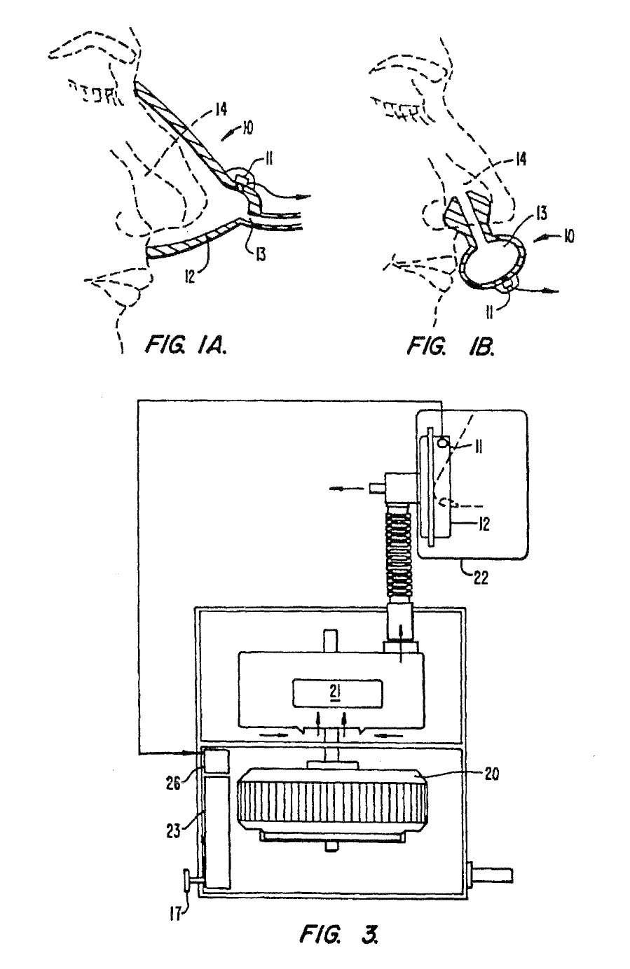

FIG. 1a illustrates a snoring detection device 10 for use in detecting triggering events for the present invention. The snoring detection device 10 comprises a microphone 11, in sound communication with the container 12 of a nose mask. Air, being inhaled by the patient, enters the nasal passageways 14 through the opening 13 in the nose mask 12 and is exhaled in the reverse direction. As the airway extends from the source of snoring sounds within the patient's body, through the nasal passages 14 and out of the opening 13 in the nasal mask, the microphone 11 is ideally located to take advantage of the natural stethoscope formed by the enclosed airway. Hence the snoring and breathing sounds are focused and concentrated by this arrangement. Alternatively, the microphone 11 may be located within, or attached externally of, a nasal prong device as illustrated in FIG. 1b. The detection device 10 can be used in a feedback control. The nose prongs and mask 12 are convenient forms of containers for the monitor device 10 however the container could take any other convenient form. Furthermore, diagnostic apparatus in accordance with the present invention is suited for use by a patient with minimal supervision and therefore may be used successfully at home.

In FIG. 3, a CPAP apparatus embodying the invention is illustrated. The CPAP unit comprises a motor 20 which drives a blower 21. The speed of the motor 20 is controlled by an electronic speed control unit 23. As an increase in motor speed also increases the blower speed which in turn increases the output air pressure of the blower 21, the speed control unit can be manipulated to vary the output pressure of the blower 21. The CPAP device also includes a snoring detection means 22 wherein sounds are detected by a microphone 11. In its most general form, the snoring detection means 22 is a pressure detection means and microphone 11 is a differential pressure sensor. The snoring detection means 22 is conveniently in the form of the previously described device 10. Electrical impulses are fed from said microphone 11 to an amplifier/filter/processor unit 26 which generates an electrical signal when snoring sounds occur. The motor speed control means is electrically connected to the snoring detection device 22 and increases the speed of the electric motor 20 by an analogue means in response to the electrical signal generated by the snoring detection device. Accordingly, the output pressure of the CPAP unit increases in response to detection of snoring.

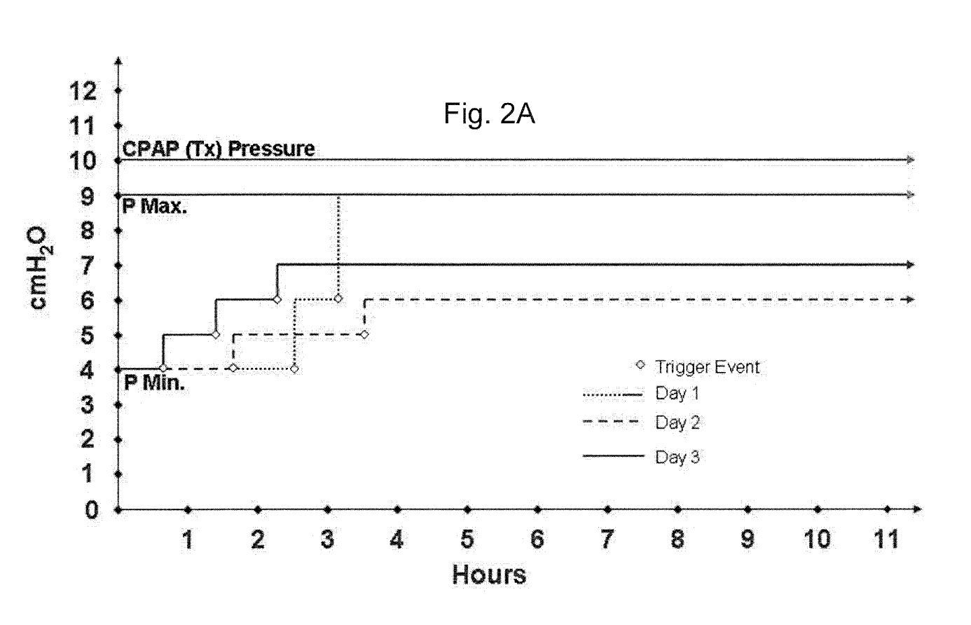

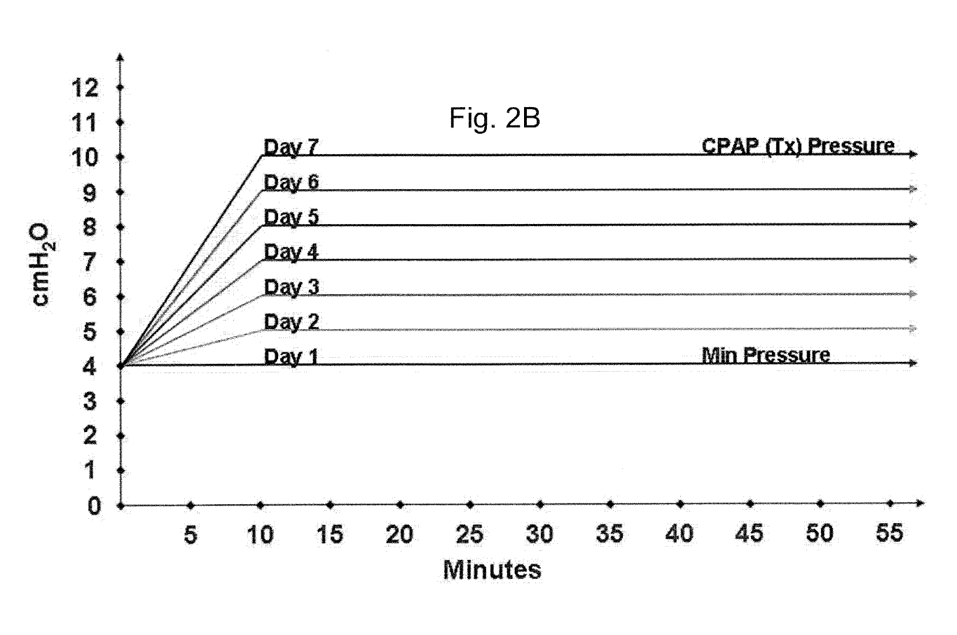

When a snore or sequence of snores is detected by the snoring detection means 22 a signal is generated. The speed control unit 23 increases the speed of the fan motor and the output pressure is increased. As snoring is caused by vibration of the soft palate, it is therefore indicative of an unstable airway and, as previously described, is a warning signal of the imminence of upper airway occlusion in patients that suffer obstructive sleep apnea. Snoring is itself undesirable not only as it is a disturbance to others but it is strongly believed to be connected with hypertension. As shown in FIG. 2A, the resultant increase in CPAP pressure is by a predetermined step value. This step may be sufficient to completely stabilize the airway, and snoring will cease. If a further snoring sound is detected, it becomes a second triggering event and the CPAP pressure is increased again by the step value. This process is repeated until the upper airway is stabilized and snoring ceases or until a predetermined maximum pressure is achieved, that pressure being bounded by a predetermined CPAP therapeutic pressure. Hence, the occurrence of obstructive apnea can be eliminated by application of minimum appropriate pressure at the time of use which varies from use to use depending upon the frequency of triggering events.

Although the invention has been described as if the step size is a predetermined constant, it may be selected in turn from a set of predetermined values so as to form a sequence of values, or be calculated from a predetermined function. The step may also be achieved by a ramp from a present pressure value to one increased by the step size.

In use, a patient may connect himself to the CPAP unit and go to sleep. The CPAP pressure is initially at a minimum operating value of, for example, approximately 3 cm H₂ O gauge pressure so as not to cause the previously mentioned operational problems of higher initial pressures. Not until some time after going to sleep, and the patient's body relaxes, will the airway start to become unstable and the patient start to snore. The detection apparatus 22 responded to a snore, or snore pattern, and via the processor 26 increase the motor speed such that CPAP pressure increases by for example 1 cm H₂ O for each snore detected. The CPAP pressure can be increased relatively rapidly, if the patient's condition so requires, to a working pressure of the order of 8-10 cm, which is a typical requirement. An upper pressure limiting device can be incorporated for safety. Also, for ease of monitoring the variation over time in patient conditions, a parameter such as pressure output could be recorded in some convenient retrievable form for periodic study by the physician. It has been found advantageous to control the rise in CPAP pressure over a longer period of time to acclimate the patient to higher pressures, but to use the occurrence of some physiologic change to trigger the increase in pressure.

If for example in the early stages of sleep some lesser CPAP pressure will suffice, the CPAP unit of the present invention will not increase the pressure until needed, that is, unless the airway becomes unstable and snoring recommences no increase is made to the airway pressure.

The flexibility of the invention can be illustrated by the following example. It is known that a patient's maximum propensity to suffer sleep apnea occurs during REM sleep. An airway that was otherwise stable at a given CPAP pressure may become unstable during REM sleep. Should this happen snoring will set in before apnea occurs. In such circumstances, the present invention will raise the CPAP pressure in response to the snoring, thus preventing the onset of apnea.

A patient normally makes at least one loud snort or snoring sound at the end of an occurrence of apnea and the present invention will respond to this unusually loud sound to increase the CPAP pressure. Thus even if apnea should occur without the usual precursor of snoring, the airway pressure can still be adjusted upward in response to the abnormally loud breathing sounds generated at the end of the apneic period.

The present invention thus provides a CPAP device which increases the CPAP pressure according to triggering events throughout an entire sleep period. It will be clear to those skilled in the art that the present invention can cope with the variation in airway pressure requirements such as may occur during a single sleep period. For subsequent sleep periods the invention returns to the same starting point, allowing each day to be independent.

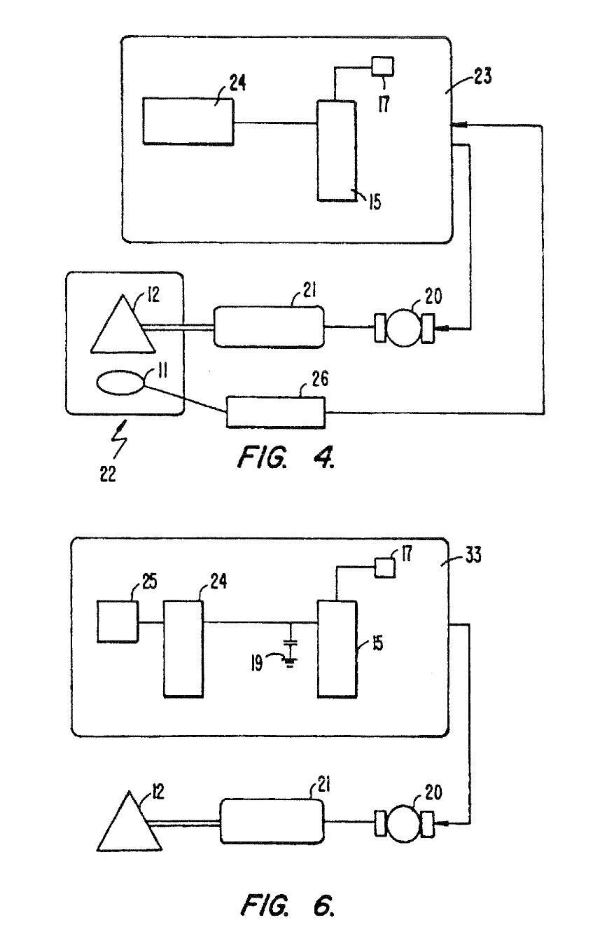

FIG. 4 illustrates in block form the circuitry of the CPAP device of FIG. 3. A snoring detection apparatus 22 is comprised of the microphone 11 attached to the nose mask 12. The electrical signals of the microphone 11 are sent to a Filter/Amplifier/Processor 26 which generates a control signal indicative of the recognition of a snoring pattern equivalent to a predetermined pattern.

Such control signals are sent to a feedback speed controller 23. The speed controller 23 comprises a ramp generator and voltage to frequency converter 24 for control of a switch mode power supply (SMPS) 15, which provides the power to run the motor 20 turning the blower 21.

The maximum output of the SMPS 15, and therefore the maximum pressure delivered to the mask 12, is limited by a pressure time control 17.

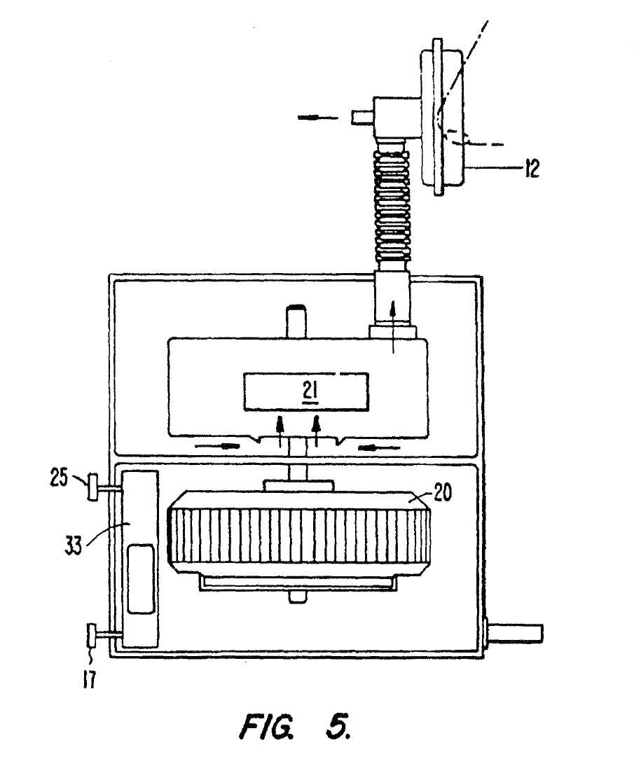

In another aspect of the invention, shown in FIGS. 5 and 6, there is provided a control circuit 33 comprising a delay control 25, a timer 24, a switch mode power supply (SMPS) 15, and an upper pressure control 17. In the timer 24 a square wave pulse train, is generated where the duty ratio can be varied by the delay control 25. This pulse train, in the form of a current, is applied to a capacitor 19 to obtain a ramp voltage. Hence the output of the timer 24 and the input of the SMPS 15 is a voltage increasing with respect to time. The output of the SMPS 15, and therefore the motor voltage and speed, follow the input.

The minimum blower speed is preset so as to give effective operation of the air blower 21 and a minimum airway pressure which is comfortable to the patient. Typically a minimum pressure of 3-5 cm H₂O will have negligible effect on most patients.

The desired maximum airway pressure, being the intended therapeutic airway pressure, is set by adjusting the variable control 17. The magnitude of this pressure will vary according to the requirements of the individual patient but will typically be in the range 10-20 cm H₂O.

When the delay control 25 is not set to zero minutes, the apparatus commences operation at the minimum motor speed and gradually increases the motor speed over a period of time selected before reaching the maximum preselected speed according to the previous adjustment of control 17. When the delay control 25 is set to zero minutes airway pressure comes up to the full level as set by adjustment 17 in a short period of time.

By this arrangement sleep is commenced with a low and comfortable air pressure but then automatically increased after a selectable period of time to the desired therapeutic pressures so as to provide an adequate pneumatic splint to the airway passages during the latter stages of sleep when apnea is likely.

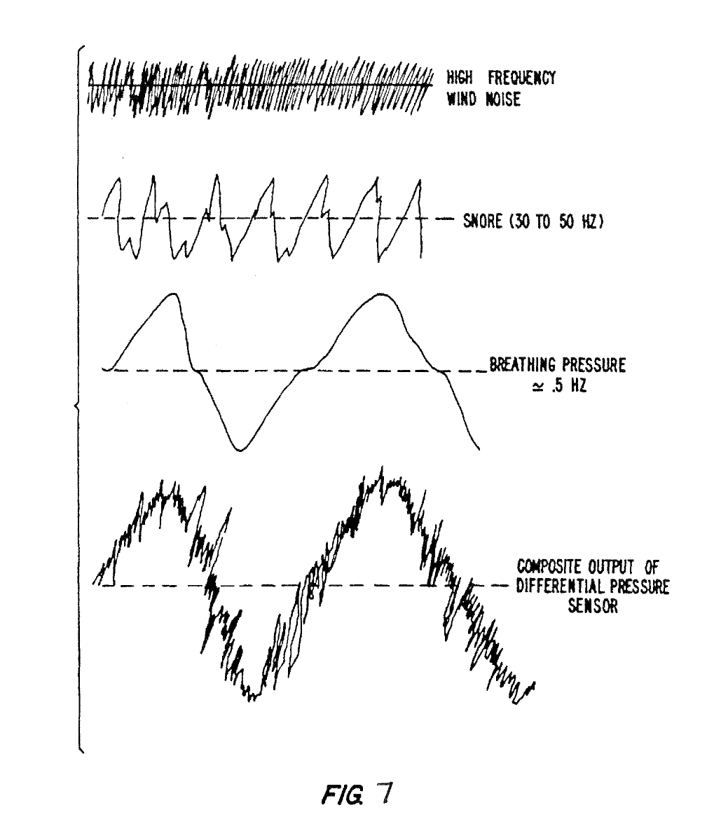

The detection device can be used in a feedback control. FIG. 7 shows the sources of pressure waves detected by the pressure sensor. The high frequency wind noise is generated by the air blower and is extraneous for the purposes of interpreting the condition of the patient. Pressure waves at a frequency of 30 to 50 Hz are indicative of snoring and could be detected by using a particular embodiment of the pressure sensor in the form of a microphone. Disturbances in breathing pattern are detected at a very low frequency relating to breathing rate of approximately 0.5 Hz. The output of the sensor is a composite of the pressure waves generated by all these sources.

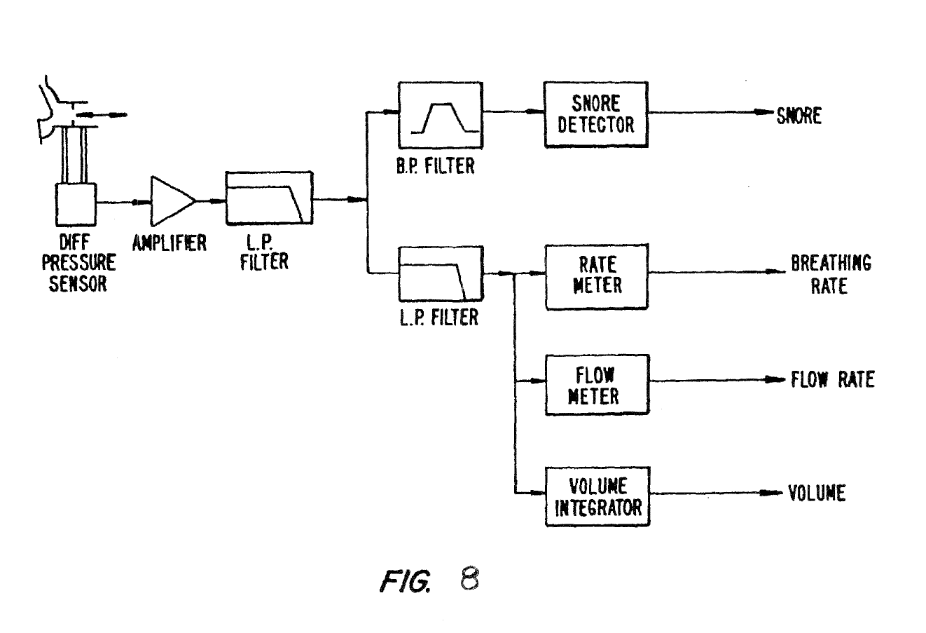

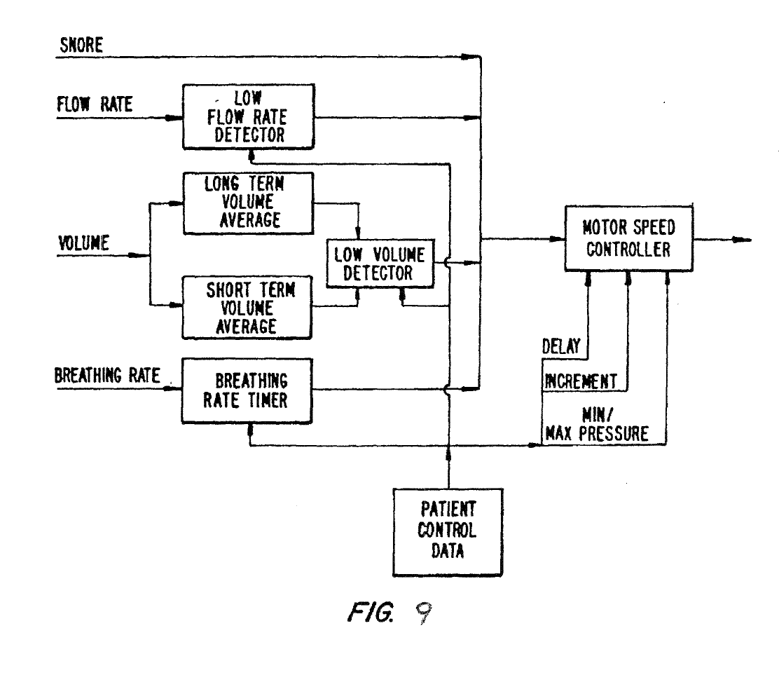

FIG. 8 shows a schematic electronic circuit which first amplifies the output of the pressure sensor before passing it through a series of filters to separate the pressure waves at the frequencies of interest. The high frequency wind noise is eliminated and signals indicative of snoring and breathing are obtained. The breathing signal is further processed to give information on breathing rate, flow rate and volume per breath.

The effect of blower motor noise can be diminished or completely removed by setting a low gain on the recording device or passing the signal through an amplitude filter to effectively ignore all sounds below a particular minimum amplitude or by passing the signals through a low pass frequency filter to effectively ignore sounds above its cutoff frequency. An alternative method is to use a sound attenuator in the air line proximate the blower.

FIG. 8 shows how the processing system can be set up to respond when the average volume of a predefined number of the most recent breaths falls below the volume of a predefined long term average volume for that patient.

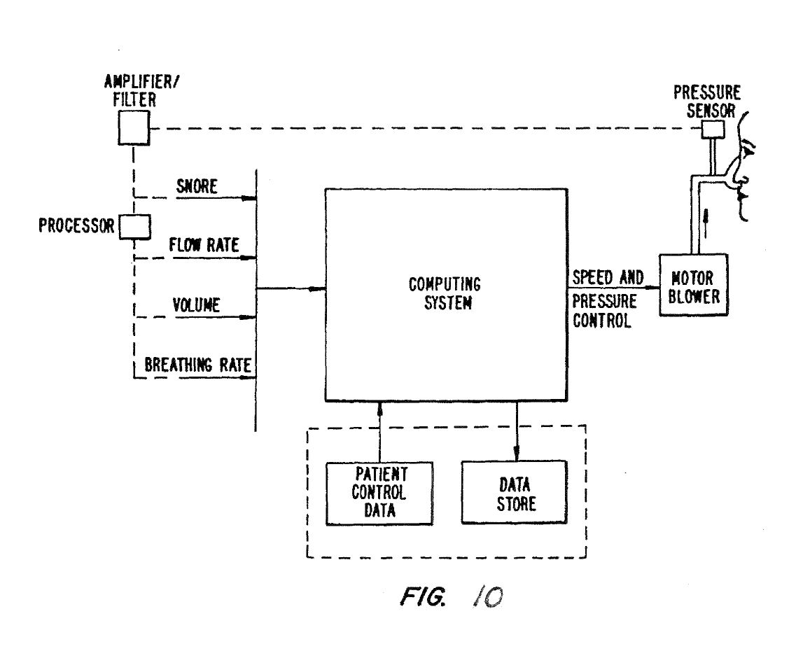

FIG. 9 shows how the computing system can accept and analyze any or all of the processed signals from the pressure sensor and using the signal, alone or by comparison with control data for that patient, control the speed of the blower to vary the pressure of the air delivered to the patient's nose mask.

Thus, the recorded information derived from the signal of the pressure sensor can be used for diagnostic purposes, such as initial diagnosis of sleep apnea or hypopnea, without the need for the patient to stay overnight in an observation facility, the sound and breathing patterns can be analyzed by a programmed microprocessor or computing system as shown in FIG. 10 so as to record tables of indexes such as number of hypopneas and/or apneic episodes, their duration and time of occurrence. This is of economic significance because the cost of one overnight observation is comparable to the purchase price of a CPAP device.

In FIG. 3, a CPAP apparatus embodying the invention is illustrated. The CPAP unit comprises a motor 20 which drives a blower 21. The speed of the motor 20 is controlled by an electronic speed control unit 23. As an increase in motor speed also increases blower speed which in turn increases the output air pressure of blower 21, the speed control unit can be manipulated to vary the output pressure of the blower 21. The CPAP device also includes a pressure detection means 22 wherein pressure waves in the form of electrical signals are detected by a pressure sensor 11. Electrical signals are fed from said sensor 11 to an amplifier/filter/processor unit 26 which may be inside or outside the blower unit casing and generates an electrical signal when snoring sound and/or deviations of breathing parameters from predetermined values occur. The motor speed control means is electrically connected to the pressure detection device 22 while being electrically isolated from the patient and increases the speed of the electric motor 20 by an analogue means in response to the electrical signal generated by the pressure detection device. Accordingly, the output, pressure of the CPAP unit increases in response to detection of snoring and/or deviations of breathing parameters from predetermined values.

The method of operation can be illustrated by considering the effect of a snore or sequence of snores detected by the pressure sensor as shown in FIG. 2a. When a snore or sequence of snores is detected by the snoring detection means 22 a signal is generated. The speed control unit 23 increases the speed of the fan motor and the output pressure is increased. As snoring is caused by vibration of the soft palate, it is therefore indicative of an unstable airway and, as previously described, is a warning signal of the imminence of upper airway occlusion in patients that suffer obstructive sleep apnea. Snoring is itself undesirable not only as it is a disturbance to others but it is strongly believed to be connected with hypertension. If the resultant increase in CRAP pressure is sufficient to completely stabilize the airway, snoring will cease. If a further snoring sound is detected, the CPAP pressure is increased again. This process is repeated until the upper airway is stabilized and snoring ceases. Hence, the occurrence of obstructive apnea can be eliminated by application of a minimum appropriate pressure at the time of use.

A predetermined deviation of any or all of the breathing parameters, flow rate, volume or breathing rate from a predetermined common value can generate a signal in a similar way. Hence a fall in the volume of air inspired or expired per breath below a preset value can generate a signal which increases the speed of the fan motor and increases the output pressure.

In use a patient may connect himself to the CPAP unit and go to sleep. Only one connection is required apart from the normal CPAP circuit and this is simply the connection from the pressure sensor to the amplifier/filter/processor unit. No electrodes or other sensors have to be attached to the patient's body as the pressure sensor is conveniently located in the CPAP mask. The CPAP pressure is initially at a minimum comfortable operating value of, for example, approximately 3 cm H₂ O gauge pressure so the as not to cause the previously mentioned operational problems of higher initial pressures. Not until some time after going to sleep, and the patient's body relaxes, will the airway start to become unstable and the patient start to snore or exhibit abnormal breathing patterns. The detection apparatus 22 will a respond to the snore, or snore pattern or abnormal breathing pattern and via the processor 26 increase the motor speed such that CPAP pressure increases by 1 cm H₂O for each snore or predetermined abnormality in breathing pattern detected. The CPAP pressure can be increased relatively rapidly, if the patient's condition so requires, to a working pressure of the order of 8-10 cm H₂O, which is a typical requirement. An upper pressure limiting device can be incorporated for safety. Also, for ease of monitoring the variation over time in patient conditions, a parameter such as pressure output can be recorded in some convenient retrievable form for periodic study by the physician.

If for example in the early stages of sleep some lesser CPAP pressure will suffice, the CPAP unit of the present invention will not increase the pressure until needed, that is, unless the airway becomes unstable and snoring or abnormal breathing patterns recommence, no increase is made to the airway pressure.

The flexibility of the invention can be illustrated by the following example.

It is known that a patient's maximum propensity to suffer sleep apnea occurs during REM sleep. An airway that was otherwise stable at a given CPAP pressure may become unstable during REM sleep. Should this happen snoring and/or particular deviations in breathing patterns will set in before apnea occurs. In such circumstances, the present invention will raise the CPAP pressure in response to the snoring or deviation in breathing patterns, thus preventing the onset of apnea or other undesirable respiratory condition. A patient normally makes at least one loud snort or snoring sound at the end of an occurrence of apnea and the present invention will respond to this unusually loud sound to increase the CPAP pressure. Thus even if apnea should occur without the usual precursor of snoring or abnormal breathing pattern, the airway pressure can still be adjusted upward in response to the abnormally loud breathing sounds generated at the end of the apneic period.

The present invention thus provides a CPAP device which modifies the CPAP pressure according to variations in a patient's requirements throughout an entire sleep period. It will be clear to those skilled in the art that the present invention can cope with the variation in airway pressure requirements such as may occur during a single sleep period, it will also be able to cope with variations in CPAP pressure requirements due to a general improvement or deterioration in a patient's general condition as may take place over a longer period of time.

FIG. 10 illustrates in block form the circuitry of the feedback system. A pressure detection apparatus is provided either integral with or attached to the CPAP mask worn by the patient. The electrical signals from the pressure transducer are amplified and filtered to provide pressure waves of the desired frequencies indicative of snoring and breathing. The pressure wave indicative of breathing is further processed to generate signals indicative of flow rate, volume and breathing rate. Any or all signals are fed to a computing system which analyses the signals for deviation from predetermined values or patterns. The computing system may itself calculate control values of patterns for each patient based on moving average values or such values or patterns will be preprogrammed into the computing system.

Where signals deviate from predetermined values or patterns the computer system generates a signal which is sent to the feedback speed controller on the blower motor. Increasing blower speed increases the air pressure and level of CPAP treatment delivered to the patient. The speed and pressure are increased until signals detected from the patient are within the acceptable range of control values or patterns and the speed and pressure are maintained at that level.

The maximum output of the blower can be limited by limiting the signal from the computer to correspond to a predetermined motor speed.

FIG. 2B depicts a sequence of pressure increases that are substantially independent of specific sleeping events for the “acclimate” mode of the present invention. Thus, instead of a triggering mechanism, a specific sequence of clocked events controls the pressure delivered to the patient. In particular, as shown in that figure, each day the pressure starts from a minimum and proceeds by a ramp to a value that increases by step from day to day until a predetermined maximum value is reached which may be the predetermined therapeutic pressure. The ramp is preferably linear, but may be some other function.

In addition, the onset of the pressure change may be delayed, allowing a predetermined period at the low pressure initial value. This permits the patient to have an initial period in which to fall asleep without any disturbance by increased pressure.

It is to be understood that while the invention has been described above in conjunction with preferred specific embodiments, the description and examples are intended to illustrate and not limit the scope of the invention, which is defined by the scope of the appended claims.

1. A method of CPAP therapy delivered to a patient comprising the steps of capturing attributes of the patient's respiration pattern over an entire sleep session, providing sealed air communication with a patient's respiratory system from an air pressure source, and delivering pressurized air from said source during said sleep session to the patient so that the air pressure automatically gradually increases over a period of time in response to triggering attributes of the patient's respiration pattern to reach a level below a predetermined maximum therapeutic operating level, wherein said period of time exceeds a single breathing cycle of the patient.

2. The method of claim 1 wherein the time period of the automatic increase exceeds an hour.

3. A method of CPAP therapy delivered to a patient comprising the steps of capturing attributes of the patient's respiration pattern over an entire sleep session, providing sealed air communication with a patient's respiratory system from an air pressure source, and delivering pressurized air from said source during said sleep session as the patient attempts to fall asleep while automatically gradually increasing said pressurized air over a period of time by a constant step magnitude to reach a operating level less than or equal to a predetermined maximum therapeutic operating level, wherein said steps vary from session to session depending upon the time and quantity of the triggering attributes of the patient's respiration pattern.

4. The method of claim 3 wherein the magnitude of said constant step can be adjusted.

5. A method of CPAP therapy delivered to a patient comprising the steps of capturing attributes of the patient's respiration pattern over an entire sleep session, providing sealed air communication with a patient's respiratory system from an air pressure source, and delivering pressurized air from said source during said sleep session to the patient so that the air pressure automatically increases by step in response to triggering attributes of the patient's respiration pattern over a period of time greater than an hour to reach a level that varies from session to session bounded above by a predetermined therapeutic operating level.

6. The method of claim 5 wherein the step size of the automatic increase is achieved in a rate of increase controlled by a ramp function.

7. A method of CPAP therapy delivered to a patient comprising the steps of capturing attributes of the patient's respiration pattern over an entire sleep session, providing sealed air communication with a patient's respiratory system from an air pressure source, and delivering pressurized air from said source during said sleep session as the patient attempts to fall asleep while automatically gradually increasing said pressurized air over a period of time to reach each day a daily level bounded above by a predetermined therapeutic operating level.

8. The method of claim 7 wherein the daily levels increase from day to day for a period of days.

9. An air flow device, useful in CPAP therapy of a patient, comprising: means for delivering variable pressure levels of pressurized air to a patient's respiratory system; means for automatic capture of the patient's breathing pattern; and means for triggering the increases to a daily level bounded above by a predetermined therapeutic operating level.

10. An air flow device, useful in CPAP therapy of a patient, comprising: a motor with a blower; a speed control unit coupled to said motor; and a control circuit coupled to said speed control, wherein said control circuit causes said blower to increase to a predetermined pressure where said means is determined based upon automatic capture of the patient's breathing pattern bounded above by a therapeutic operating level wherein said increase occurs over a period of days

11. The device of claim 10 wherein said control circuit may be set to adjust the maximum increase for any one day.

12. The device of claim 11 wherein said control circuit may be set to adjust the therapeutic operating level.

13. The device of claim 12 wherein said control circuit commences operation at a minimum pressure.

14. The method of claim 1 wherein said triggering attribute is recognition of cessation of breathing.

15. The method of claim 3 wherein said triggering attribute is recognition of cessation of breathing.

16. The method of claim 5 wherein said triggering attribute is recognition of cessation of breathing.

17. A method of CPAP therapy comprising the steps of:
providing sealed air communication with a patient's respiratory system from an air pressure source,
delivering pressurized air from said source to the patient,
gradually increasing the air pressure over several sleep periods in response to triggering or clocked events to reach a level below a predetermined maximum therapeutic operating level, and
detecting abnormal breathing patterns of the patient during and promptly varying the air pressure in accordance with detected abnormal breathing patterns during each sleep period.

18. The method of claim 17, wherein the sleep period is defined by a period of time equal to one or more days.

19. The method of claim 18, wherein the detected abnormal breathing patterns include the average volume of air of a predefined number of patient breaths decreasing below the volume of air of a predefined long term average air volume for the patient.

20. The method of claim 19, wherein the onset of all changes to delivered air pressure in a single sleep period are delayed for a predefined period of time to allow patient to fall asleep without disturbance from increased air pressure.

21. A method of CPAP therapy comprising the steps of: providing sealed air communication with a patient's respiratory system from an air pressure source, and delivering pressurised air from said source to the patient so that the air pressure automatically gradually increases over several sleep periods in response to triggering or clocked events to reach a level below a predetermined maximum therapeutic operating level; and further wherein, said the onset of all changes to delivered air pressure in a single sleep period are delayed for a predefined period of time to allow patient to fall asleep without disturbance from increased air pressure.

22. The method of claim 21, wherein the sleep period is defined by a period of time equal to one or more days.

23. The method of claim 22, wherein the detected abnormal breathing patterns include the average volume of air of a predefined number of patient breaths decreasing below the volume of air of a predefined long term average air volume for the patient.

24. An airflow device, useful in CPAP therapy, said device comprising:
a pressure delivery system for delivering pressure levels of pressurized air to a patient's respiratory system,
a triggering or clocked event sensor providing signals to said pressure delivery system wherein the pressure of said pressurized air is gradually increased over several sleep period to reach a level below a predetermined maximum therapeutic operating level,
an abnormal breathing pattern detector providing signals to said pressure delivery system wherein, in response to said signals from said abnormal breathing pattern detector the pressure of said pressurized air is instantaneously varied during a single sleep period.

25. The device of claim 24, wherein the device comprises: a motor with a blower; a speed control unit coupled to said motor; and a control circuit coupled to said speed control, wherein said control circuit varies amount of air pressure delivered by the device.

26. The device of claim 25, wherein the sleep period is defined by a period of time equal to one or more days.

27. The device of claim 26, wherein the detected abnormal breathing patterns include the average volume of air of a predefined number of patient breaths decreasing

28. The device of claim 27, wherein the onset of all changes to delivered air pressure in a single sleep period are delayed for a predefined period of time to allow patient to fall asleep without disturbance from increased air pressure.

29. An airflow device, useful in CPAP therapy, said device comprising:
a pressure delivery system for delivering pressure levels of pressurized air to a patient's respiratory system,
a triggering or clocked event sensor providing signals to said pressure delivery system wherein the pressure of said pressurized air is gradually increased over several sleep period to reach a level below a predetermined maximum therapeutic operating level,
a clock providing a clock signal at a predefined period of time to said pressure delivery system wherein the onset of all changes to delivered air pressure in a single sleep period are delayed for a predefined period of time to allow the patient to fall asleep without disturbance from increased air pressure.

30. The device of claim 29, wherein the device comprises: a motor with a blower; a speed control unit coupled to said motor; and a control circuit coupled to said speed control, wherein said control circuit varies amount of air pressure delivered by the device.

31. The device of claim 30, wherein the sleep period is defined by a period of time equal to one or more days.

32. The device of claim 31, wherein the detected abnormal breathing patterns include the average volume of air of a predefined number of patient breaths decreasing below the volume of air of a predefined long term average air volume for the patient.
